(19) United States
(12) Patent Application Publication
(10) Pub. No.: US 2002/0165506 A1
(43) Pub. Date: Nov. 7, 2002

(54) Individual breast wipes (disposable)

(76) Inventors: Carolyn Jane Smith, North Charleston, SC (US); Virgil Smith JR., North Charleston, SC (US)

Correspondence Address: CAROLYN J. SMITH, 104 LAUREL RIDGE ROAD, NORTH CHARLESTON, SC, 29418, US

(21) Appl. No.: 09/848,517

(22) Filed: May 4, 2001

(51) Int. Cl. A61M 35/00

(52) U.S. Cl. 604/289

(57) ABSTRACT

Individual Breast Wipes are handy moistened absorbent wipes that will clean the breast nipple area of mothers in preparation for breast-feeding. The function of the wipes will eliminate the problems of mothers after delivery. It will give them the assurance that the breast is clean and that they do not have to get out of bed to get a paper towel to clean their breast after delivery.

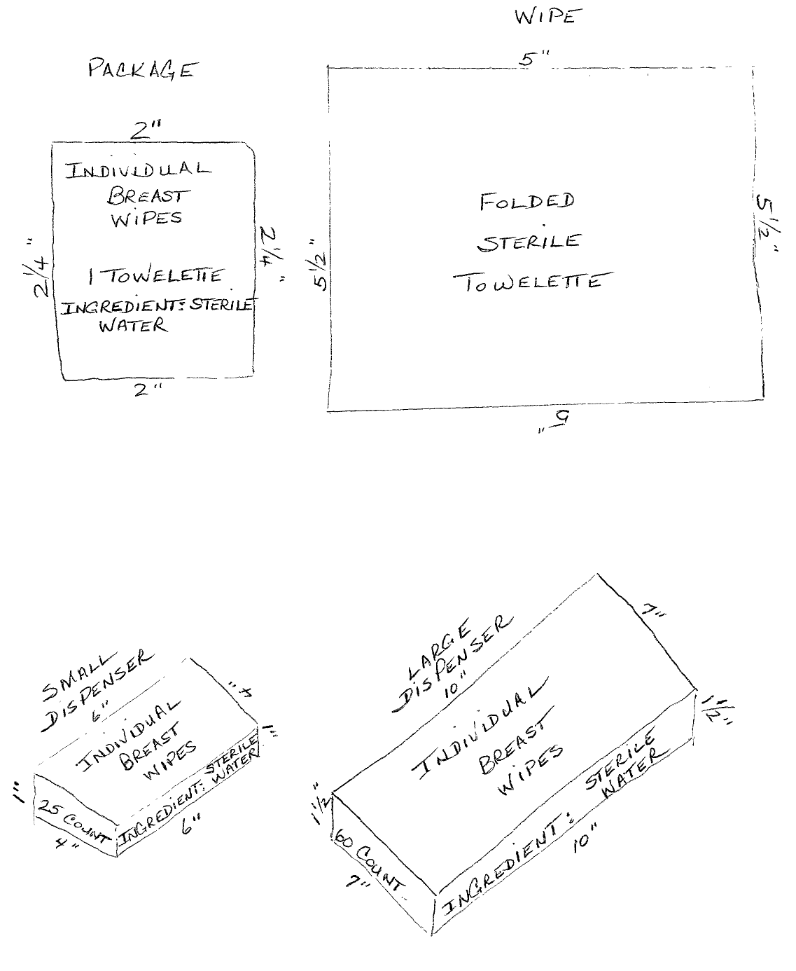

### DETAILED DESCRIPTION OF THE INVENTION

Individual Breast Wipes are soft towelettes moistened with sterilized water. The sterile towelettes are used to clean the area of the breast for infant feeding. The nipples of the mother's breast and the area of the breast the baby's mouth will touch. Two or three towelettes Breast Wipes could clean the entire breast. The babies of nursing mothers will places their little hands all over the breast. The nursing mother will feel good about her clean breast to feed her new baby.

Individual Breast Wipes are all folded into one package, two inches by two and one-quarter inches. The towelette wipes would be five inches by five and one-half inches. The ingredient in every package will be sterilized water.

After the breast-feeding is done, Breast Wipes can be used to clean all teething rings, baby bottle nipples, baby toys, and their little hands.

Small dispensers of Breast Wipes will have twenty-five Breast Wipes. Large Breast Wipes Dispenser will have sixty Breast Wipes.

We claim:

1. Individual Breast Wipes (disposable)
Individual Breast Wipes Towelette (5 ½″×5″)
Individual Breast Wipes Package (2 ¼″×2″)
One Individual Breast Wipe Towelette will be in each package.
One small dispenser (25 count) will be 6″×4″×1″.
One large dispenser (60 count) will be 10″×7″×1 ½″.
